(19) United States
(12) Patent Application Publication
(10) Pub. No.: US 2026/0100194 A1
(43) Pub. Date: Apr. 9, 2026

(54) Systems and Methods for Detecting Subvocalization

(71) Applicant: Cirrus Logic International Semiconductor Ltd., Edinburgh (GB)

(72) Inventors: John P. LESSO, Edinburgh (GB); Yanto SURYONO, Tokyo (JP); Toru IDO, Kanagawa (JP)

(73) Assignee: Cirrus Logic International Semiconductor Ltd., Edinburgh (GB)

(21) Appl. No.: 19/261,716

(22) Filed: Jul. 7, 2025

Related U.S. Application Data
(60) Provisional application No. 63/705,086, filed on Oct. 9, 2024.

(51) Int. Cl. G10L 15/25; A61B 5/00; A61B 5/11; G10L 15/08; G10L 25/78

(52) U.S. Cl. CPC G10L 15/25; A61B 5/1114; A61B 5/4542; G10L 15/08; G10L 25/78; A61B 2503/12

(57) ABSTRACT

A method of processing subvocalized speech of a user, the method comprising: receiving an audio signal from an input transducer configured to capture speech of the user; receiving a first motion signal from a first motion sensor configured to detect motion of a jaw or a temporomandibular joint (TMJ) of the user; determining the presence of audible speech in the audio signal; when audible speech is present in the audio signal: correlating the audible speech with first motion data in the first motion signal to generate mapping data mapping the first motion data to the audible speech; when audible speech is not present in the audio signal: determining subvocalised speech of the user based on the first motion data and the mapping data.

### TECHNICAL FIELD

The present disclosure relates to methods and systems for determining the content of subvocalised speech.

### BACKGROUND

Speech recognition technology has traditionally depended on audible inputs captured by microphones to transcribe spoken words into text or to execute voice commands. However, reliance on audible speech poses limitations in certain situations, such as where silence is necessary, where background noise is overwhelming, or for individuals with speech impairments.

### SUMMARY

According to a first aspect of the disclosure, there is provided a method of processing subvocalized speech of a user, the method comprising: receiving an audio signal from an input transducer configured to capture speech of the user; receiving a first motion signal from a first motion sensor configured to detect motion of a jaw or a temporomandibular joint (TMJ) of the user; determining the presence of audible speech in the audio signal; when audible speech is present in the audio signal: correlating the audible speech with first motion data in the first motion signal to generate mapping data mapping the first motion data to the audible speech; when audible speech is not present in the audio signal: determining subvocalised speech of the user based on the first motion data and the mapping data.

According to another aspect of the disclosure, there is provided a method of processing subvocalized speech of a user, the method comprising: receiving a first motion signal from a first motion sensor configured to detect motion of a jaw or a temporomandibular joint (TMJ) of the user; receiving a second motion signal from a second motion sensor configured to detect motion of a jaw or a temporomandibular joint (TMJ) of the user, the second motion sensor spaced apart from the first motion sensor; determining asymmetric motion of the jaw based on a differential signal derived from the first and second motion signals; and determining subvocalised speech of the user based on the determined asymmetric motion.

The following option features may apply to one or both of the above-mentioned aspects, where applicable.

The method may further comprise transcribing the audible speech.

The method may further comprise identifying one or more acoustic classes of the audio speech.

The one or more acoustic classes of speech may comprise one or more of the following: phonemes; plosives; sibilants; affricates; fricatives; vowels; and approximants.

Correlating the audible speech with the first motion data comprises mapping each of the identified one or more acoustic classes to motion data temporally aligned with the respective identified one or more acoustic classes.

Correlating the audible speech with the first motion data may comprise implementing one or more machine learning algorithms or trained classifiers to correlate the audio speech with the motion data and to classify motion patterns into the determined subvocalised speech.

Correlating the audible speech with the first motion data may comprise providing the audio signal and the motion signal as inputs to a trained neural network, the trained neural network configured to predict the subvocalised speech based on the motion data.

The method may further comprise pre-processing the audio signal and/or the first motion signal using one or more low-pass filters or Kalman filters.

The method may further comprise receiving a second motion signal from a second motion sensor configured to detect motion of a jaw or a temporomandibular joint (TMJ) of the user, the second motion sensor spaced apart from the first motion sensor.

The method may further comprise correlating the audible speech with second motion data in the second motion signal with to generate the mapping data mapping the second motion data to the audible speech. The subvocalised speech may be determined based on the second motion data.

The method may further comprise generating a difference signal representing a difference between the first and second motion signals; and correlating audible speech with asymmetric motion data in the difference signal, the asymmetric motion data representing asymmetric motion of the jaw, wherein the subvocalised speech is determined based on the asymmetric motion data.

The first motion sensor may be mechanically coupled to a first side of the user's head, and the second motion sensor may be mechanically coupled to a second side of the user's head.

The audio signal may comprise one or more audio prompts for enrolment of the user.

The method may further comprise, when audible speech is not detected, outputting an output audio signal to an audio output transducer.

The output audio signal may comprise an indication of the determined subvocalised speech.

During an enrolment stage, the output audio signal may comprise one or more prompts to the user to recite one or more predetermined words or phrases.

The one or more prompts may include prompts to the user to recite the one or more predetermined words or phrases using subvocalised speech. The method may further comprise: correlating the motion data with the predetermined words or phrases.

The method may further comprise determining a quality metric related to the motion data; and if the quality metric is below a predetermined quality threshold, discarding the motion data for correlation with the audible speech.

The quality metric may comprise a signal to noise ratio of the motion data.

Determining the presence of audible speech in the audio signal may comprise receiving an additional audio signal from an additional input transducer configured to detect bone conducted speech of the user; and correlating the additional audio signal with to the audio signal.

The method may further comprise outputting the determined subvocalised speech to a speech processor.

The speech processor may be configured to perform speech command analysis, speech recognition, dictation, and/or voice assistant functionality.

The speech processor may comprise an applications processor or a central processing unit.

The motion sensor may comprise an inertial measurement unit.

The motion sensor may comprise an electromyography sensor configured to detect muscle activity associated with motion of the jaw.

The input transducer and the motion sensor may be integrated into a personal device.

The motion sensor may be mechanically coupled to the user proximate the jaw or TMJ.

The receiving may be performed at a wearable device worn by the user. One or more of the determining the presence of audible speech, correlating, and determining subvocalised speech may be performed by a host device in communication with the wearable device.

The method may further comprise determining symmetric motion of the jaw based on a common mode signal derived from the first and second motion signals; and determining the subvocalised speech of the user based on the determined symmetric motion.

The first motion sensor may be positioned at or proximate a left side of the jaw, and the second motion sensor is positioned at or proximate a right side of the jaw.

According to another aspect of the disclosure, there is provided a wearable device for processing subvocalized speech of a user, the personal device comprising: an input transducer to generate an audio signal capturing speech of the user; a first motion sensor configured to generate a first motion signal capturing motion of a jaw or a temporomandibular joint (TMJ) of the user; processing circuitry configured to: determining the presence of audible speech in the audio signal; when audible speech is present in the audio signal: correlate the audible speech with first motion data in the first motion signal to generate mapping data mapping the first motion data to the audible speech; when audible speech is not present in the audio signal: determine subvocalised speech of the user based on the first motion data and the mapping data.

According to another aspect of the disclosure, there is provided a wearable device for processing subvocalized speech of a user, the personal device comprising: a first motion sensor configured to generate a first motion signal in response to motion of a jaw or a temporomandibular joint (TMJ) of the user; a second motion sensor configured to generate a second motion signal in response to motion of a jaw or a temporomandibular joint (TMJ) of the user, the second motion sensor spaced apart from the first motion sensor; and processing circuitry configured to: determine asymmetric motion of the jaw based on a differential signal derived from the first and second motion signals; and determine subvocalised speech of the user based on the determined asymmetric motion.

The wearable device may comprise a headset, a headphone, an earbud, an earphone, augmented reality glasses, virtual reality glasses, or a smart watch.

According to another aspect of the disclosure, there is provided a system comprising: the wearable device described above, and a host device, wherein the host device comprises one of a smartphone, a personal computer, a laptop computer, a tablet computer, a smart watch.

According to another aspect of the disclosure, there is provided a method of processing subvocalized speech of a user, the method comprising: receiving an audio signal from an input transducer configured to capture speech of the user; receiving a first motion signal from a first motion sensor configured to detect motion of a jaw or a temporomandibular joint (TMJ) of the user; determining subvocalised speech of the user based on the first motion data and the mapping data mapping the first motion data to the audible speech.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the present disclosure will now be described by way of non-limiting examples with reference to the drawings, in which:

FIG. 1 is a schematic diagram of a personal wearable device;

FIG. 2 is a schematic diagram of the personal wearable device;

FIGS. 3 and 4 are schematic diagram of example implementations of a classifier implemented by a processor of the personal wearable device or a host device coupled to the personal wearable device;

FIGS. 5 and 6 graphically illustrate discrimination of jaw movements associated with certain words of vocalised and subvocalised speech; and

FIGS. 7 and 8 are confusion charts graphically illustrating the accuracy of prediction during training and testing modes of operation.

### DESCRIPTION OF EMBODIMENTS

Embodiments of the present disclosure relate to apparatus, methods and systems for speech recognition based on the motion of a speaker's jaw.

A transducer of a personal audio device, such as an inertial measurement unit (or IMU) in an earbud, can be used to detect jaw motion during speech by measuring subtle changes in the position and orientation of the skull and ear of the speaker caused by jaw movements. These changes in position and orientation are translated by the IMU into corresponding motion patterns, enabling real-time tracking of speech-related jaw motion. Jaw motion detection may be most effective for detecting speech actions which are particularly tied to jaw positioning. Examples of acoustic classes of speech which exhibit such speech actions include plosives, vowels, affricates, and nasals, with vowels being more particularly tied to jaw positioning. In contrast, sounds (specifically phones) such as glottals, sibilants, fricatives, approximants, and high vowels, may be less detectable through jaw motion due to associated speech actions in volving minimal jaw movement. Such sounds are more reliant on the tongue, lips, or vocal folds of the speaker, and are therefore harder to detect.

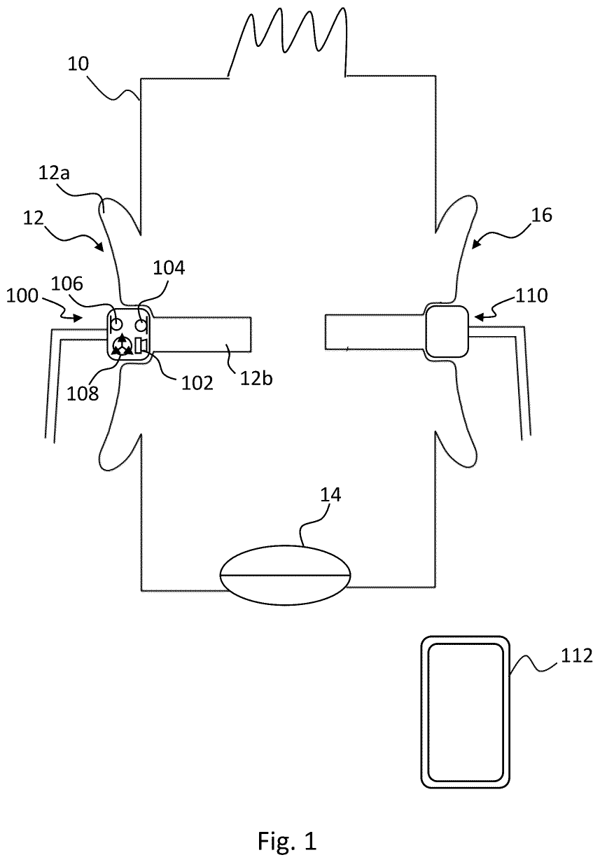

FIG. 1 shows a schematic diagram of a user's ear 12 and mouth 14. The user's ear 12 comprises an (external) pinna or auricle 12a, and the (internal) ear canal 12b.

A personal device comprising an intra-concha headphone 100 (or earphone) sits inside the user's concha cavity. The headphone 100 may fit loosely within the cavity, allowing the flow of air into and out of the user's ear canal 12b which results in partial occlusion of the ear canal of the user. Alternatively, the headphone 100 may form a tight seal with the ear canal which may result in full occlusion.

The headphone 100 comprises one or more loudspeakers 102 positioned on an internal surface of the headphone 100 and arranged to generate acoustic signals towards the user's ear and particularly the ear canal 12b. The headphone 100 may further comprise one or more microphones 104, known as error microphone(s) or internal microphone(s), positioned on an internal surface of the earphone, arranged to detect acoustic signals within the internal volume defined by the headphone 100 and the ear canal 12b. The headphone 100 may also comprise one or more microphones 106, known as reference microphone(s) or external microphone(s), positioned on an external surface of the headphone 100 and configured to detect environmental noise (or air-conducted sound) incident at the user's ear.

The headphone 100 may be able to perform active noise cancellation (ANC), to reduce the amount of noise experienced by the user of the headphone 100 as is known in the art.

The headphone 100 further comprises an inertial measurement unit (IMU) 108, comprising an accelerometer and/or a gyroscope, configured to measure inertia at or proximate the jaw of the user 10. In this case, the IMU 108 is located in the headphone 100 which is placed in or on the ear 12. However, the IMU 108 may be placed elsewhere on the head of the user 10, provided it can detect movement of the user's speech articulators (such as the jaw, ear, brow, nose etc.). The present disclosure is not limited to the provision of an IMU 108. In other embodiments, the IMU 108 may be replaced with another motion sensor capable of detecting motion of speech articulators of the user 10. An example alternative motion sensor is an electromyography (EMG) sensor. EMG sensors may be configured to detect muscle activity (e.g. movement) associated with articulation of the jaw of the user 10.

With the headphone 100 located in or at the user's ear 12, the IMU 108 is configured to detect motion of the user's jaw or TMJ when the user is speaking (vocalised speech) or miming speech (subvocalised speech). Subvocalised speech may be completely silent or may comprise unvoiced speech. In either case, motion of the user's jaw or TMJ is translated by the IMU 108 into a motion signal which can be used for training of a speech model as well as determining what a user is saying when mining or subvocalising speech. The IMU 108 may comprise one or more accelerometers and/or one or more gyroscopes. Accelerometer data obtained from an accelerometer may be used to detect linear displacement of the jaw or TMJ. Gyroscope data may be used to detect rotational movement of the jaw or TMJ. The combination of a linear component of jaw displacement and rotational movement may then be used to characterise movements associated with speech.

Due to the fit of the headphone 100 in or on the ear 12, the IMU 108 may be mechanically coupled to the user's head. As such, the IMU 108 may be configured to pick up sound associated with voiced speech of the user 10 conducted through the user's head via a bone-conduction path of the user's head, so-called bone-conducted speech and/or speech conducted through soft tissue and cartilage

In the example shown in FIG. 1, an intra-concha headphone 100 is provided as an example personal device. It will be appreciated, however, that embodiments of the present disclosure can be implemented on any personal audio device which is configured to be placed at, in or near the ear of a user or on the head of the user. Examples include circum-aural headphones worn over the ear, supra-aural headphones worn on the ear, in-ear headphones inserted partially or totally into the ear canal to form a tight seal with the ear canal, or mobile handsets held close to the user's ear so as to provide audio playback (e.g. during a call). Embodiments of the present disclosure may be implemented in any type of personal device that comprises a microphone and an IMU or other movement sensor. Examples include virtual reality headsets, augmented reality headsets and smart glasses to name a few. In such examples, an IMU (such as the IMU 108) may be positioned at any location on the head, such as the jaw, provided they are able to pick up movement of the jaw relative to the head, or the TMJ.

The headphone 100 may form part of a headset comprising another headphone 110 configured in substantially the same manner as the headphone 100. For example, like the headphone 100, the headphone 110 may comprise one or more of an error microphone, a reference microphone, a loudspeaker, and an accelerometer. The other headphone 110 may be configured to be placed in or on another ear 16 of the user 10. The pair of headphones 100, 110 may form a stereo headset.

The headphone 100 and the other headphone 110 (when provided) may be in wired or wireless communication with a host device 112. The host device 112 may be a personal device, such as a smartphone or personal computer such as a laptop or tablet computer.

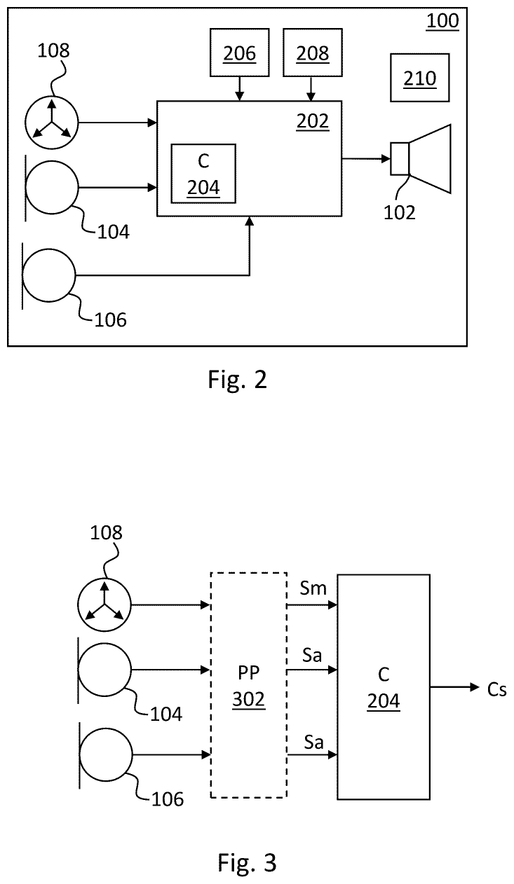

FIG. 2 is a system schematic of the headphone 100.

A signal processor 202 of the headphone 100 is configured to receive microphone signals from the microphones 104, 106 and the IMU 108 and output audio signals to the loudspeaker 102. The headphone 100 may be configured for a user to listen to music or audio, to make telephone calls, to deliver voice commands to a voice recognition system, and/or other such audio processing functions. The processor 202 may be configured to implement active noise cancellation (feedback and/or feedforward) and/or passthrough/transparency modes using the microphones 104, 106 and the one or more transducers 102. The processor 202 is also configured to obtain biometric data from the IMU 108 and/or the one or more microphones 104, 106, as will be explained in more detail below. The biometric data may pertain to the user's voice (via the one or more microphones 104, 106) in addition to the user's jaw motion when speaking (via the IMU 108). The processor 202 may be configured to implement a classifier 204. Operation of the classifier 204 will be described in detail below.

The headphone 100 further comprises a memory 206, which may in practice be provided as a single component or as multiple components. The memory 206 is provided for storing data and/or program instructions. The headphone 100 further may further comprise a transceiver 208, which is provided for allowing the headphone 100 to communicate (wired or wirelessly) with external devices, such as the other headphone 110, and/or the host device 112 to which the headphone 100 is coupled. Such communications between the headphone 100 and external device(s) may comprise wired communications where suitable wires are provided between left and right sides of a headset, either directly such as within an overhead band, or via an intermediate device such as a mobile device and/or wireless communications. The headphone may be powered by a battery 210 and may comprise other sensors (not shown). It will be appreciated that methods described herein may be implemented on the headphone 100 or on the host device 112 to which the headphone 100 is connected, or a combination of both.

As mentioned above, the IMU 108 may be comprise an accelerometer, and/or a gyroscope, in either two-axis or three-axis configurations. The IMU 108 may be configured to output inertial measurements to the processor 202. The IMU 108 may form part of the headphone 100 as shown in FIG. 1. Alternatively, the IMU 108 may be a separate module in communication with the headphone 100, for example, via the transceiver 208. In some embodiments, for example where the headphone 100 is implemented as a headset worn on a user's head, the IMU 108 may be positioned away from the ear of the user when worn.

The IMU 108 may be used to generate one or more signals representative of motion of the headphone 100 which may be used as a proxy for motion of the jaw of a user 10. Examples of motion include linear displacement (e.g. movement forward, back, left, right, up, down etc) as well as rotation or tilt in any direction. A change in movement or tilt may also be derived from signals received from the IMU 108. For example, signals output from the IMU 108 may be pre-processed by the processor 202 before being processed by the classifier 204. Pre-processing by the processor 202 may comprise filtering. Such filtering may be tuned for the speed at which the jaw can physically move or rotate. For example, signals obtained from the IMU 108 may be filtered with a low-pass filter or a Kalman filter. Such filters may account for signal discontinuities associated with non-physical jaw motion (e.g. avoiding a signal interpretation of a jaw instantly jumping from fully opened to fully closed).

Vibrations due to speech of the user, conducted via the bone-conduction path in the user's head, may also be picked up by the IMU 108. Thus, the IMU 108 may be used to determine one or more characteristics of the user's speech. The IMU 108 may therefore be used instead of or in addition to the one or more microphones 104, 106 to detect speech of the user 10.

The one or more microphones 104, 106 may be used to monitor for the presence of audible speech. In doing so, motion data from the IMU 108 may be correlated with speech content or characteristics, as described further below.

As noted above, the headphone 100 may be configured for the user 10 to listen to music or audio, to make telephone calls, to deliver voice commands to a voice recognition system, and/or other such audio processing functions. As such loudspeaker 102 may be configured to output audio content. Such audio content may be for generation of audio prompts to enable enrolment, and/or to playback determined subvocalised speech content, to allow a user to confirm that their subvocalised speech has been correctly identified, e.g. by the processor 202. For example, the prompts may comprise prompts to the user to recite one or more predetermined words or phrases. For example, the prompts may comprise prompts to the user to recite such predetermined words or phrases using subvocalised speech.

Embodiments of the present disclosure utilise motion signals from the IMU 108 to generate a model of how the user's 10 jaw moves during speech. This model can be trained during periods in which the user 10 is speaking audibly (vocalised speech) or speaking with subvocalised speech as prompted (e.g. predetermined words or phrases). Audible speech can be picked up by one or both microphones 102, 104 and/or via the IMU 108 through bone conduction through the head of the user 10. By combining linear displacement and gyroscopic data, a full picture of how the jaw of the user 10 moves during speech production can be ascertained.

Classification of speech based on jaw motion may be performed by correlating certain jaw movements with different speech sounds. In general, speech may be classified in terms of distinct phonemes. A phoneme is a distinct unit of sound in a specific language that can change the meaning of a word. Phonemes are language-specific and represent abstract categories of sounds. For example, in English, /p/ and /b/ are phonemes because they distinguish words like “pat” and “bat.”

Jaw motion of the user 10 is detectable via an IMU 108 due to the physical movements that occur during speech articulation. The jaw (mandible) is connected to the skull through the temporomandibular joint (TMJ), located near the ears. When the jaw of the user 10 moves, whether to open or close their mouth 14, these movements also slightly shift the position of the surrounding bones and tissues, including areas near the ear canal. Such motion is described in “Forensic Speaker Identification”, Phil Rose, ISBN: 978-0415271820, the contents of which is hereby incorporated by reference in its entirety.

When the jaw of the user 10 moves, these shifts are transferred to the area around the ear. This can cause slight movements of the skull, ear cartilage, and the earbud itself. As noted throughout, this movement can be picked up by the IMU 108 whether located at the ear, or in the vicinity of the ear.

Jaw movements during speech, such as opening for vowels or closing for plosives, create distinct motion patterns. The IMU 108 can capture these movements as the jaw drops (downward acceleration) and raises (upward acceleration). Certain speech sounds, particularly lateral approximants (like /l/) or more complex consonants, can involve slight side-to-side motion of the jaw. When the IMU 108 comprises a gyroscope, the IMU 108 can pick up this rotational motion, providing additional data about jaw dynamics. The movements associated with producing plosives (like /p/, /t/, /k/) involve rapid closure and release of the jaw, which would be picked up by an accelerometer and gyroscope of the IMU 108, when provided. Different speech sounds, such as vowels and plosives, create distinct motion profiles. For instance, a vowel like /a/ involves a larger, more sustained jaw drop compared to a consonant like /t/, which involves a quick closure and release. The IMU 108 can be used to track these differences.

Thus, the headphone 100 (or other device) may preferably have a portion which is located proximate the TMJ. The TMJ is the primary joint responsible for jaw movement. Movements of the jaw, whether small or large, translate to changes in the TMJ, and these changes propagate to the ear. Since the headphone 100 is sitting in or near the ear canal, it is located in a preferable location to detect these movements. Since the ear is directly connected to the skull and jaw via bone, there's less damping or absorption of motion compared to sensors placed on other parts of the body, such as the wrists or chest. It will be appreciated, however, that sensor placed anywhere on the head of the user 10 may provide the necessary signals required to detect jaw motion.

Jaw motion correlates with several families of speech sounds, particularly those that involve significant oral articulation. These include plosives, vowels, nasals, and affricates, explained in more detail below.
Plosives (Stop Consonants). Examples: /p/, /b/, /t/, /d/, /k/, /g/. These sounds involve a complete closure of the vocal tract followed by a burst of air. The jaw often moves to create the closure, especially for /p/, /b/, /k/, and /g/. Jaw lowering is especially notable during the release of these sounds.
Vowels Examples: /a/, /e/, /i/, /o/, /u/. Vowel production is strongly linked to jaw motion. Low vowels like /a/ and [symbol] / (as in “hot”) involve the jaw dropping significantly, while high vowels like /i/ and /u/ (as in “see” and “blue”) require less jaw opening.
Nasals Examples: /m/, /n/, / [symbol] /. These sounds are produced by closing the oral cavity but allowing air to pass through the nasal cavity. The jaw often moves slightly to facilitate closure, especially in /m/ and / [symbol] /.
Affricates. Examples: /t∫/ (as in “ch”), /d [symbol] / (as in “judge”). Jaw Movement: Affricates are combinations of a stop followed by a fricative. They involve significant jaw motion, particularly for /t∫/ and /d [symbol] /.

Sounds that involve minimal or subtle jaw movement that are more challenging to detect through jaw motion alone include glottal sounds, sibilants and fricatives, approximants, and high vowels, as explained in more detail below.
Glottal Sounds Examples: /h/ (as in “hat”), /?/ (glottal stop). These sounds are produced in the glottis (the space between the vocal cords) and involve little to no movement in the mouth or jaw. The primary articulation happens at the vocal folds, making jaw motion less relevant for detection.
Sibilants and Fricatives Examples: /s/, /z/, /∫/ (as in “sh”), / [symbol] / (as in “measure”), /f/, /v/, /θ/ (as in “thin”), /ð/ (as in “this”). These sounds are characterized by the turbulent airflow through a narrow constriction in the vocal tract, often involving the tongue and lips. While there may be slight jaw movement, it is usually subtle, and the primary articulators are the tongue or lips rather than the jaw.
Approximants Examples: /j/ (as in “yes”), /w/ (as in “wet”), /r/ (as in “red”). Approximants involve the narrowing of the vocal tract, but the constriction is less tight than in fricatives. The jaw motion is minimal because these sounds primarily depend on the positioning of the tongue and lips.
Lateral Approximant Example: /l/ (as in “light”). The sound is created by allowing the airflow to pass around the sides of the tongue, with relatively little involvement of the jaw. The tongue is the primary articulator here.
High Vowels Examples: /i/ (as in “see”), /u/ (as in “blue”). High vowels are produced with the tongue raised high in the mouth, and the jaw remains mostly closed. The jaw movement for these vowels is minimal compared to low vowels like /a/.

The following table provides further examples of phrases that may be challenging to detect using jaw motion alone, i.e. detecting plosives, vowels, affricates, and nasals but not detecting fricatives, sibilants, approximants, and glottal sounds.

| Words | What makes them hard to detect via jaw motion? |
|---|---|
| “bat” vs. “sat” vs. | The distinction between these words comes from the sounds |
| “fat” vs. “that” | /b/, /s/, /f/, and /δ/ (as in “that”). /b/ is a plosive, so it would be |
| | detectable. |
| | However, /s/, /f/, and /δ/ are fricatives, which rely on tongue and |
| | lip |
| | positioning without much jaw movement, making them hard to |
| | detect. |
| “dog” vs. “log” | The distinction is between /d/ (plosive) and /l/ (approximant). |
| | You could detect the plosive /d/, but recognizing the |
| | difference between “dog” and “log” would be challenging due |
| | to the approximant /l/ relying on tongue |
| | position rather than jaw movement. |
| “she” vs. “see” | Problem: The distinction is between /∫/ (as in “she,” a sibilant) |
| | and /s/ (as |
| | in “see”). Both are sibilants and involve little jaw motion, so |
| | detecting the difference between these two words would be |
| | difficult. |
| “sip” vs. “zip” vs. | The distinction between these words comes from /s/, /z/, and /t∫/ |
| “chip” | (as in “chip,” an affricate). While the affricate /t∫/ might be |
| | detectable, you would struggle to distinguish /s/ from /z/, since |
| | both are sibilant fricatives that |
| | involve minimal jaw motion |
| “veil” vs. “fail” | The key difference is between /v/ (fricative) and /f/ (fricative). |
| | These two sounds are distinguished by the position of the lips |
| | and teeth, not by jaw |
| | motion, making them hard to distinguish. |
| “wed” vs. “led” | The difference between /w/ (approximant) and /l/ (lateral |
| | approximant) is subtle and mainly relies on tongue and lip |
| | positioning. These are difficult to detect without detecting fine |
| | articulatory movements other than jaw |
| | motion. |

An analysis of the detectability of certain common commands used for controlling a personal device using jaw motion alone is provided in the following table.

| Word | Detectability | Sounds | Notes |
|---|---|---|---|
| Skip | Partially | /s/ (fricative), | You would likely detect the /k/, /p/, |
| | detectable | /k/ (plosive), | and vowel /I/, but the /s/ (fricative) |
| | (missing /s/). | /I/ (vowel), | might be harder to detect. |
| | | /p/ (plosive) | |
| Stop | Partially | /s/ (fricative) | You can detect the /t/, /p/, and the |
| | detectable | /t/ (plosive) | vowel /a/, but /s/ (fricative) would be |
| | (missing /s/) | /a/ (vowel) | difficult. Similar to “skip,” you could |
| | | /p/ (plosive) | detect part of the word, but missing |
| | | | the/s/ sound could cause confusion |
| | | | with other words. |
| Up | Fully | /Λ/ (vowel) | This word is easy to detect since it |
| | detectable | /p/ (plosive) | consists of a vowel and a plosive, |
| | | | both of which are easily tracked |
| | | | through jaw |
| | | | motion. |
| Down | Fully | /d/ (plosive) | You can detect /d/, the diphthong /a [symbol] / |
| | detectable. | /a [symbol] / (vowel | (vowel), and /n/ (nasal). |
| | | diphthong) | |
| | | /n/ (nasal) | |
| Play | Partially | /p/ (plosive) | You can detect the /p/ and the |
| | detectable | /l/ (approximant), | diphthong /eI/, but /l/ (approximant) |
| | (missing | /eI/ (vowel | would be difficult to detect. Partial |
| | /l/). | diphthong) | detection, but it might be confused |
| | | | with words like “pay.” |
| Pause | Partially | /p/ (plosive) | You can detect /p/ and / [symbol] / (vowel), but |
| | detectable | / [symbol] / (vowel), | /z/ (fricative) is difficult. Partial |
| | (missing | /z/ (fricative) | detection, but missing the final /z/ |
| | /z/). | | could cause confusion with other |
| | | | words like |
| | | | “paw.” |
| Siri | Difficult to | /s/ (fricative) | You can detect the vowels /I/ and /i/, |
| | detect | /I/ (vowel) | but /s/ (fricative) and /r/ (approximant) |
| | (missing | /r/ (approximant), | would be hard to detect. This word |
| | /s/ and /r/). | /i/ (vowel) | would be difficult to detect in its |
| | | | entirety. |

It will be understood, therefore, that it may be easier to determine subvocalised speech corresponding to some audible sounds than others. For example, plosives, vowels, affricates and nasals can be easily tracked in jaw motion due to the relatively large jaw movements associated with these sounds.

Phonemes which can be easily detected, such as plosives, vowels, affricates, and nasals, cover many of the most prominent components of human speech in the English language. For example, plosives and affricates are associated with stops and some complex consonants. For example, vowels can be detected form the core of syllables and are essential for distinguishing between words. For example, nasals are present in many words and can provide important phonetic information.

Whilst some sounds can be captured using in motion using the IMU 108 or the one or more microphones 104, 106, subvocalized speech can involve subtle internal articulations within the mouth 14 which do not result in actual air release or external sound. For example, certain phonemes such as Fricatives and Sibilants are challenging to detect via jaw motion. Many common sounds such as /s/, /z/, /∫/ (as in “sh”), and /f/ would be difficult to track reliably based on jaw motion alone. These sounds are useful in distinguishing many words in English and other languages. For example, detecting the difference between “sat” and “fat” would be problematic. Glottal sounds like /h/ or glottal stops, which involve movements of the vocal cords rather than the jaw, would not be detectable. Approximants such as /l/, /r/, and /w/ often involve minor jaw movement but are primarily dependent on tongue and lip positioning. These sounds also carry important distinctions in many words.

While plosives and vowels account for much of the energy in speech, speech recognition relies heavily on fine distinctions made by less jaw-influenced sounds like fricatives and approximants. If such sounds cannot be detected, it may be difficult to distinguish words that have similar plosive/vowel combinations but differ by subtle consonants. For example, words like “bat” vs. “vat” or “pat” vs. “sat” could be hard to distinguish.

Thus, to improve the accuracy of detection, the classifier 204 (or processor 202) may combine jaw-motion tracking with other context-based information. Such context-based information may comprise knowledge of likely word sequences, for example using language models such as Hidden Markov Models (HMMs).

In addition, even if the subvocalization involves only certain elements of speech, such as plosive sounds or certain vowels, partial detection may still provide useful information upon which a determination of speech characteristics can be obtained.

In the examples described above, a single headphone 100 is used in infer speech based on jaw motion. However, embodiments are not so limited. For example, embodiments of the present disclosure also provide a system and method for detecting unvoiced speech or subvocalized speech using two or more spaced apart transducers mechanically coupled to the head of the user 10. For example, one of the transducers (e.g. IMU 108) of the headphone 100 and another transducer of the other headphone 110 may be used to monitor motion. The outputs of the two of more spaced apart transducers may be used to determine symmetric and asymmetric motion of the jaw of the user 10. The unvoiced speech or subvocalized content may be determined based on both the symmetric and asymmetric motion of the jaw as determined by the combination of signals from separated transducers.

Thus, referring to FIGS. 1 and 2, signals from IMUs 108 of the headphones 100, 110 may be processed together, e.g. by the processor 202 or the host device 112 inferring motion of the jaw of the user. It will be appreciated, once again, that any wearable or mountable device may be used to implement the system described herein.

The IMU 108 and the IMU of the other headphone 110 may collectively output common mode signals, differential signals, or both. Each or both of these signals may be used to identify symmetric and asymmetric jaw motion. This is distinct from conventional headset implementations in which a common mode signal is typically used for detecting both bone conducted speech and head movement.

In more detail, when the IMU 108 (and the IMU of the other headphone 110) are positioned on either side of the head of the user 10, the differential signal between the output signals of the IMUs can capture asymmetrical movements of the jaw, particularly those that involve lateral (side-to-side) or rotational motion. Various sounds have an asymmetric sound component (for example, see “Vowels and Consonants”, Peter Ladefoged et al., ISBN: 978-1444334296, the contents of which is hereby incorporated by reference in its entirety). Examples of such sounds are as follows.
Plosives (Stop Consonants) Examples. /p/, /b/, /t/, /d/, /k/, /g/. Plosives involve significant jaw movement, especially during the closure and release of air. The bilateral difference between the two sides of the jaw, particularly for sounds like /k/ and /g/ (which involve the back of the tongue), could generate a detectable asymmetry.
Vowels Examples: /a/, /e/, /o/ (low or mid vowels). For low and mid vowels (e.g., /a/as in “father”), the jaw drops significantly, and slight asymmetries in jaw movement (depending on speech articulation) might be detected. However, high vowels like /i/ and /u/ generally do not involve enough lateral movement for a strong difference signal.
Affricates: Examples. /t∫/ (as in “ch”), /d [symbol] / (as in “judge”). These involve both a stop and a fricative component, leading to significant jaw motion. The closure and release stages may create asymmetry that can be detected, especially during the transition from the stop to the fricative.
Nasals Examples. /m/, /n/, / [symbol] / Nasal sounds involve oral cavity closure and can generate some asymmetry in jaw positioning, particularly for sounds like / [symbol] / (as in “sing”) where the back of the tongue and soft palate are involved.

The difference signal between the two IMUs would be most sensitive to sounds that involve asymmetrical jaw motion, particularly plosives, nasals, affricates, and certain vowels (mainly low and mid vowels). These sounds involve greater lateral or rotational movement in the jaw, leading to detectable differences between the two IMUs. In contrast, fricatives, approximants, high vowels, and glottal sounds would generate little to no difference signal because they involve minimal jaw asymmetry or lateral motion.

Processing of the one or more microphone signals 104, 106 and the IMU 108 of the headphone 100, in addition to corresponding signals from the other headphone 110 will now be described. Such processing may be performed by the processor 220, a processor of the other headphone 110, the host device 112, or by a remote device coupled to the host device 112, such as a server or in the cloud. In the following description, processing is implemented by the processor 202 of the headphone 100, which is configured to implement the classifier 204.

The classifier 204 may implement a trained neural network, such as a convolutional neural network. The neural network may be trained to identify speech content or characteristics based on monitored motion data of the jaw or TMJ from one or more motion detecting sensors. For example, the neural network may be trained with inputs relating to motion and speech of the user. The trained neural network may then be used to predict speech characteristics based on motion of the user's jaw or head as detected by motion detecting sensors. Implementations of neural networks are known in the art and so will not be described in detail here. The motion detecting sensors may be the IMU 108 of the headphone 100 and/or sensors of the other headphone 110. Additionally, or alternatively, the neural network may be trained using generic motion and speech data. The classifier 214 may be arranged to output determined speech content or characteristics.

In any of the examples described herein, the processor 202 may determine a quality metric associated with motion data obtained from the IMU 108. For example, the processor 202 may determine a signal to noise ratio (SNR) of signals obtained from the IMU 108. If the quality metric is below a predetermined threshold, corresponding motion data associated with that low quality metric may be discarded or flagged as being below the predetermined threshold. When discarded, that motion data may then not be used for learning or training of any classification described herein.

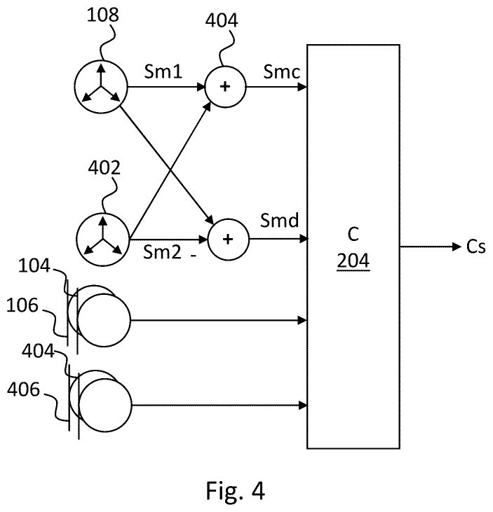

FIG. 3 is a schematic diagram of an example implementation of the classifier 204 for receiving signals from the headphone 100. The classifier 204 is configured to receive one or more motion signal Sm from the IMU 108 in addition to one or more audio signals Sa from one or both of the microphones 104, 106. Optionally, the one or more motion and audio signals Sm, Sa are pre-proceed using one or more filters (e.g. low pass filters or Kalman filters). The classifier 204 is configured to process the one or more motion and audio signals Sa, Sm (filtered or otherwise) and output one or more speech characteristics Cs.

When audio is detected in the one or more audio signals Sa, Sm, the classifier 204 may be configured in a learning mode in which a correspondence between speech and jaw movement of the user 10 may be learnt. The processor 202 may determine that speech in the one or more audio signals Sa, Sm is speech of the user (as opposed to speech of a third party) by correlating speech from the external microphone 106 with speech from the internal microphone 104 (if both are provided). A correlation may indicate that the user is speaking since speech sound received at the internal microphone 104 via a bone conduction path would be correlated to speech sound received at the external microphone 106.

In the learning mode, speech-to-text conversion may be used to transcribe audible speech into text. In parallel, motion monitoring is used to capture motion data from the IMU 108. The transcribed speech data may then be correlated with the motion data to learn motion patterns corresponding to speech content.

When audio is not detected in the one or more audio signals Sa, Sm, the classifier 204 may be configured in an inference mode in which the speech characteristic Cs may be output from the classifier 204. The speech characteristic Cs may then comprise an estimate of speech which corresponds to one or more motion signals Sm (which itself corresponds to certain jaw movement).

For example, motion detection captures motion data from the IMU 108. The motion signal Sm is used to determine motion of the jaw or the temporomandibular joint (TMJ). The classifier 204 (implementing e.g. a neural network trained on the learned correlation between motion data and speech) is then configured to determine unvoiced or subvocalized speech content based on the monitored jaw or TMJ movement. The determined speech content may be output to a speech processor for the handling of user speech, e.g. via speech command or dictation systems.

The classifier 204 may rely on certain relationships between certain combinations of movements as picked up in the one or more motion signals Sm and the corresponding audible speech sound to which they relate. Such relationships have been described in detail above.

FIG. 4 is a schematic diagram of an example implementation of the classifier 204 for receiving signals from the headphone 100 and the other headphone 110.

The output of the two or more transducers can be combined to determine asymmetric jaw motion, based on a difference between motion signals from the IMU 108 and an IMU 402 of the headphone 110. Symmetric motion may be determined based on the common-mode signal from the outputs of the two IMUs 108, 402 to determine symmetric jaw motions. It will be understood that the output of a single transducer may be used to determine some degree of asymmetric motion. For example, in some implementations where the IMUs 108, 402 are mounted at the left and right ears 12, 16 of the user 10, the IMUs 108, 402 may provide common-mode and differential outputs as well as the isolated outputs from the left and right transducers.

Thus, in FIG. 4, the classifier 204 is configured to receive a common mode motion signal Smc which is the sum of a first motion signal Sm1 from the IMU 108 of the headphone 100 and a second motion signal SM2 from an IMU 402 of the other headphone 110. The first and second motion signal Sm1, Sm2 are added at an adder 404 to obtain the common mode motion signal Smc. The classifier is further configured to receive a difference motion signal Smd which represents the difference between the first motion signal Sm1 and the second motion signal Sm2. The second motion signal Sm2 is subtracted from the first motion signal Sm1 by a subtractor 406 to obtain the difference motion signal Smd. The classifier 204 may additionally receive one or more audio signals Sa from one or both of the microphones 104, 106 of the headphone 100 and/or one or both microphones 408, 410 of the other headphone 110. Optionally, the one or more motion and audio signals Sm1, Sm2, Sa are pre-proceed using one or more filters (e.g. low pass filters or Kalman filters) (not shown). The classifier 204 is configured to process the one or more motion and audio signals Smc, Smd, Sa (filtered or otherwise) and output one or more speech characteristics Cs as with the arrangement in FIG. 3.

In some embodiments, the common mode motion signal Smc may not need to be derived. Instead, the first motion signal Sm1 may be provided to the classifier 204 and the adder 404 omitted.

Like the implementation of the classifier 204 in FIG. 3, the classifier 204 may operate in a learning or training mode (e.g. when audible speech is detected) and a recognition or inference mode (e.g. when audible speech is not present).

The symmetric and asymmetric motion data derived from the differential and common mode motion signals Smd, Smc can be used to determine unvoiced or subvocalized speech content. It will be understood that the symmetric and asymmetric motion may be provided as an input to the classifier 204 for both training and inference/recognition. The classifier 204 may be trained based, in part, on the two-mode operation as described above, or may be trained in any other manner, e.g. via offline training alone.

The classifier 204 may additionally be trained to recognize non-speech-related jaw movements, e.g. chewing, yawning, and to filter out or classify such movements as not related to subvocalized speech.

The described systems and methods may be implemented in a wearable device preferably mechanically coupled with a user jaw or TMJ, e.g. a headset, earbuds, earphones, AR/VR glasses. While the processor 202 and classifier 204 may be implemented within the headphone 100 or the headphone 110 or other wearable device, such devices may be coupled with the host device 112, e.g. a cell phone, a laptop computer, a tablet computer, a smart watch, wherein the processor 202 and/or classifier 204 may be implemented in the host device 112. It will be understood that the wearable device may be coupled with the host device 112 via any suitable wired or wireless connection.

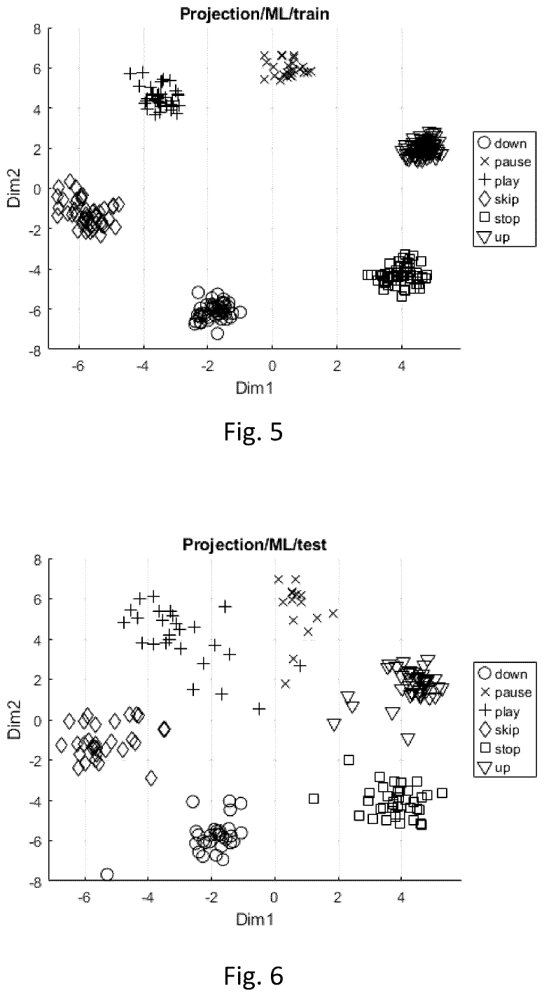

FIGS. 5 and 6 illustrate the detectability of unvoiced or whispered speech based on the outputs of the IMU 108. Linear Discriminative Analysis (or LDA) was used to learn different classes from motion signals obtained from the IMU 108. The two most significant vectors for discriminating voice commands such as “down”, “pause”, “play”, “skip”, “stop”, and “up” are plotted against each other in FIG. 5.

FIG. 6 shows the discrimination between certain voice commands based on motion signals from the IMU 108 alone. Again, the two most significant vectors for discriminating the above voice commands are plotted. It can be seen that different voice commands can be identified using just two vectors.

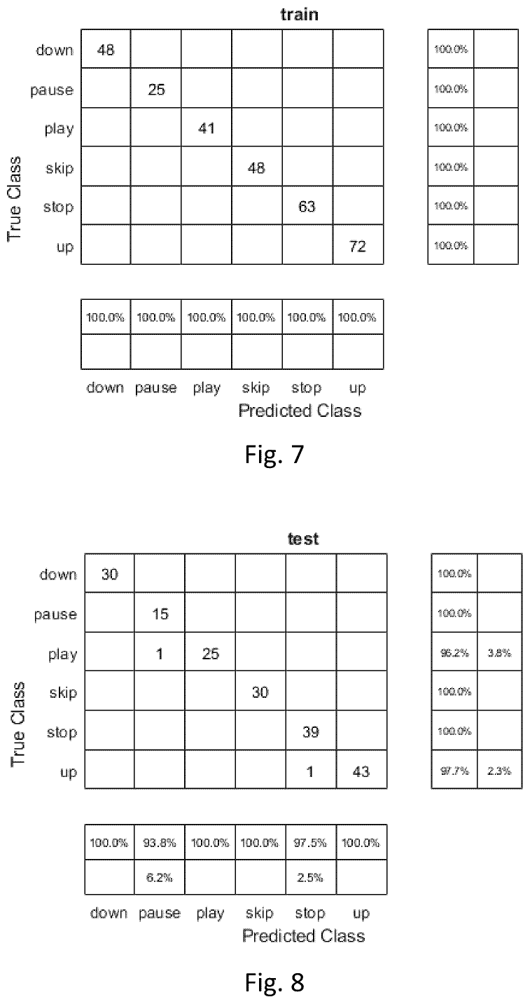

The efficacy of this jaw motion sensing approach is further illustrated in FIGS. 7 and 8 which are confusion charts comparing training and test predictions.

FIG. 7 illustrates a 100% accuracy of detection of class of sound during a training or learning mode.

FIG. 8 illustrates a high degree of accuracy during testing using a different (newly seen) dataset. Of the 26 samples tested, only two were incorrectly classified.

Optionally, the IMU acceleration vector can be rotated such that the gravity vector to in the Z-axis (by convention). Further optionally, the data may be converted to spherical co-ordinates or quaternions (Quaternions to be preferred to avoid the gimbal lock problem).

The skilled person will recognise that some aspects of the above-described apparatus and methods may be embodied as processor control code, for example on a non-volatile carrier medium such as a disk, CD- or DVD-ROM, programmed memory such as read only memory (Firmware), or on a data carrier such as an optical or electrical signal carrier. For many applications embodiments of the invention will be implemented on a DSP (Digital Signal Processor), ASIC (Application Specific Integrated Circuit) or FPGA (Field Programmable Gate Array). Thus, the code may comprise conventional program code or microcode or, for example code for setting up or controlling an ASIC or FPGA. The code may also comprise code for dynamically configuring re-configurable apparatus such as re-programmable logic gate arrays. Similarly, the code may comprise code for a hardware description language such as Verilog TM or VHDL (Very high-speed integrated circuit Hardware Description Language). As the skilled person will appreciate, the code may be distributed between a plurality of coupled components in communication with one another. Where appropriate, the embodiments may also be implemented using code running on a field-(re)programmable analogue array or similar device in order to configure analogue hardware.

Note that as used herein the term module shall be used to refer to a functional unit or block which may be implemented at least partly by dedicated hardware components such as custom defined circuitry and/or at least partly be implemented by one or more software processors or appropriate code running on a suitable general-purpose processor or the like. A module may itself comprise other modules or functional units. A module may be provided by multiple components or sub-modules which need not be co-located and could be provided on different integrated circuits and/or running on different processors.

Embodiments may be implemented in a host device, especially a portable and/or battery powered host device such as a mobile computing device for example a laptop or tablet computer, a games console, a remote control device, a home automation controller or a domestic appliance including a domestic temperature or lighting control system, a toy, a machine such as a robot, an audio player, a video player, or a mobile telephone for example a smartphone.

As used herein, when two or more elements are referred to as “coupled” to one another, such term indicates that such two or more elements are in electronic communication or mechanical communication, as applicable, whether connected indirectly or directly, with or without intervening elements.

This disclosure encompasses all changes, substitutions, variations, alterations, and modifications to the example embodiments herein that a person having ordinary skill in the art would comprehend. Similarly, where appropriate, the appended claims encompass all changes, substitutions, variations, alterations, and modifications to the example embodiments herein that a person having ordinary skill in the art would comprehend. Moreover, reference in the appended claims to an apparatus or system or a component of an apparatus or system being adapted to, arranged to, capable of, configured to, enabled to, operable to, or operative to perform a particular function encompasses that apparatus, system, or component, whether or not it or that particular function is activated, turned on, or unlocked, as long as that apparatus, system, or component is so adapted, arranged, capable, configured, enabled, operable, or operative. Accordingly, modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

Although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described above.

Unless otherwise specifically noted, articles depicted in the drawings are not necessarily drawn to scale.

All examples and conditional language recited herein are intended for pedagogical objects to aid the reader in understanding the disclosure and the concepts contributed by the inventor to furthering the art and are construed as being without limitation to such specifically recited examples and conditions. Although embodiments of the present disclosure have been described in detail, it should be understood that various changes, substitutions, and alterations could be made hereto without departing from the spirit and scope of the disclosure.

Although specific advantages have been enumerated above, various embodiments may include some, none, or all of the enumerated advantages. Additionally, other technical advantages may become readily apparent to one of ordinary skill in the art after review of the foregoing figures and description.

It should be noted that the above-mentioned embodiments illustrate rather than limit the invention, and that those skilled in the art will be able to design many alternative embodiments without departing from the scope of the appended claims. The word “comprising” does not exclude the presence of elements or steps other than those listed in a claim, “a” or “an” does not exclude a plurality, and a single feature or other unit may fulfil the functions of several units recited in the claims. Any reference numerals or labels in the claims shall not be construed so as to limit their scope.

1. A method of processing subvocalized speech of a user, the method comprising:
receiving an audio signal from an input transducer configured to capture speech of the user;
receiving a first motion signal from a first motion sensor configured to detect motion of a jaw or a temporomandibular joint (TMJ) of the user;
determining the presence of audible speech in the audio signal;
when audible speech is present in the audio signal:
correlating the audible speech with first motion data in the first motion signal to generate mapping data mapping the first motion data to the audible speech;
when audible speech is not present in the audio signal:
determining subvocalised speech of the user based on the first motion data and the mapping data.

2. The method of claim 1, further comprising:
transcribing the audible speech.

3. (canceled)

4. The method of claim 1, further comprising identifying one or more acoustic classes of the audio speech, wherein the one or more acoustic classes of speech comprise one or more of the following:
phonemes;
plosives;
sibilants;
affricates;
fricatives;
vowels; and
approximants.

5. The method of claim 4, wherein correlating the audible speech with the first motion data comprises mapping each of the identified one or more acoustic classes to motion data temporally aligned with the respective identified one or more acoustic classes.

6. The method of claim 1, wherein correlating the audible speech with the first motion data comprises implementing one or more machine learning algorithms or trained classifiers to correlate the audio speech with the motion data and to classify motion patterns into the determined subvocalised speech.

7. The method of claim 1, wherein correlating the audible speech with the first motion data comprises providing the audio signal and the motion signal as inputs to a trained neural network, the trained neural network configured to predict the subvocalised speech based on the motion data.

8. The method of claim 1, further comprising:
pre-processing the audio signal and/or the first motion signal using one or more low-pass filters or Kalman filters.

9. The method of claim 1, further comprising:
receiving a second motion signal from a second motion sensor configured to detect motion of a jaw or a temporomandibular joint (TMJ) of the user, the second motion sensor spaced apart from the first motion sensor.

10. The method of claim 9, further comprising:
correlating the audible speech with second motion data in the second motion signal to generate the mapping data mapping the second motion data to the audible speech, wherein the subvocalised speech is determined based on the second motion data.

11. The method of claim 9, further comprising:
generating a difference signal representing a difference between the first and second motion signals;
correlating audible speech with asymmetric motion data in the difference signal, the asymmetric motion data representing asymmetric motion of the jaw, wherein the subvocalised speech is determined based on the asymmetric motion data.

12. The method of claim 9, wherein the first motion sensor is mechanically coupled to a first side of the user's head, and the second motion sensor is mechanically coupled to a second side of the user's head.

13. The method of claim 1, wherein the audio signal comprises one or more audio prompts for enrolment of the user.

14. The method of claim 1, further comprising:
when audible speech is not detected, outputting an output audio signal to an audio output transducer.

15. The method of claim 14, wherein the output audio signal comprises an indication of the determined subvocalised speech.

16. The method of claim 14, wherein, during an enrolment stage, the output audio signal comprises one or more prompts to the user to recite one or more predetermined words or phrases.

17. The method of claim 16, wherein the one or more prompts include prompts to the user to recite the one or more predetermined words or phrases using subvocalised speech, the method further comprising:
correlating the motion data with the predetermined words or phrases.

18. The method of claim 1, further comprising:
determining a quality metric related to the motion data; and
if the quality metric is below a predetermined quality threshold, discarding the motion data for correlation with the audible speech.

19. (canceled)

20. The method of claim 1, wherein determining the presence of audible speech in the audio signal comprises:
receiving an additional audio signal from an additional input transducer configured to detect bone conducted speech of the user; and
correlating the additional audio signal with to the audio signal.

21. The method of claim 1, further comprising:
outputting the determined subvocalised speech to a speech processor.

22.-23. (canceled)

24. The method of claim 1, wherein the motion sensor comprises an inertial measurement unit.

25. (canceled)

26. The method of claim 1, wherein the input transducer and the motion sensor are integrated into a personal device.

27. (canceled)

28. The method of claim 1, wherein the receiving is performed at a wearable device worn by the user, and wherein one or more of the determining the presence of audible speech, correlating, and determining subvocalised speech is performed by a host device in communication with the wearable device.

29. A method of processing subvocalized speech of a user, the method comprising:
receiving a first motion signal from a first motion sensor configured to detect motion of a jaw or a temporomandibular joint (TMJ) of the user;
receiving a second motion signal from a second motion sensor configured to detect motion of a jaw or a temporomandibular joint (TMJ) of the user, the second motion sensor spaced apart from the first motion sensor;
determining asymmetric motion of the jaw based on a differential signal derived from the first and second motion signals; and
determining subvocalised speech of the user based on the determined asymmetric motion.

30.-31. (canceled)

32. A wearable device for processing subvocalized speech of a user, the personal device comprising:
an input transducer to generate an audio signal capturing speech of the user;
a first motion sensor configured to generate a first motion signal capturing motion of a jaw or a temporomandibular joint (TMJ) of the user;
processing circuitry configured to:
determine the presence of audible speech in the audio signal;
when audible speech is present in the audio signal:
correlate the audible speech with first motion data in the first motion signal to generate mapping data mapping the first motion data to the audible speech;
when audible speech is not present in the audio signal:
determine subvocalised speech of the user based on the first motion data and the mapping data.

33. (canceled)

34. The wearable device of claim 32, wherein the wearable device comprises a headset, a headphone, an earbud, an earphone, augmented reality glasses, virtual reality glasses, or a smart watch.

35. A system comprising:
the wearable device of claim 32; and
a host device, wherein the host device comprises one of a smartphone, a personal computer, a laptop computer, a tablet computer, a smart watch.

36. A method of processing subvocalized speech of a user, the method comprising:
receiving an audio signal from an input transducer configured to capture speech of the user;
receiving a first motion signal from a first motion sensor configured to detect motion of a jaw or a temporomandibular joint (TMJ) of the user;
determining subvocalised speech of the user based on the first motion data and the mapping data mapping the first motion data to the audible speech.
